(19) United States
(12) Patent Application Publication
(10) Pub. No.: US 2004/0220583 A1
(43) Pub. Date: Nov. 4, 2004

(54) Instrumentation for total knee arthroplasty, and methods of performing same

(75) Inventors: Joseph W. Pieczynski II, Austin, TX (US); Charles H. Perrone JR., Austin, TX (US)

Correspondence Address: J. Mike Amerson, Williams, Morgan & Amerson, P.C., 10333 Richmond, Suite 1100, Houston, TX, 77042, US

(73) Assignee: Zimmer Technology, Inc.

(21) Appl. No.: 10/771,871

(22) Filed: Feb. 4, 2004

Related U.S. Application Data
(60) Provisional application No. 60/444,901, filed on Feb. 4, 2003.

(51) Int. Cl. A61B 17/58

(52) U.S. Cl. 606/102

(57) ABSTRACT

The present invention is directed to instrumentation for total knee arthroplasty, and methods of performing same. In one illustrative embodiment, the device is adapted to be coupled to a prepared end of a femur and comprises a body having a bottom surface and a movable stylus operatively coupled to the body, the stylus having a tip, wherein the stylus is coupled to the body such that the tip may be moved in a direction that is approximately perpendicular to a plane containing the bottom surface. In one illustrative embodiment of the present invention, the method comprises making an incision in a patient's knee and attaching a femoral implant sizing guide to a prepared surface of a femur of the patient, the sizing guide having a body having a bottom surface and a movable stylus with a tip. The method further comprises, after the sizing guide is attached to the prepared surface of the femur, moving the tip of the stylus in both a direction that is approximately perpendicular to a plane containing the bottom surface of the sizing device and in a direction that is approximately parallel to the plane containing the bottom surface to position the tip of the stylus at a location proximate an anterior cortex region of the femur and determining a size of a femoral knee prosthesis to be positioned on the femur.

### PRIORITY CLAIM

Applicants hereby claim priority based upon U.S. Provisional Patent Application Serial No. 60/444,901, filed Feb. 4, 2003, entitled “Total Knee Arthroplasty,” the entirety of which is hereby incorporated by reference.

### BACKGROUND OF THE INVENTION

1. Field of the Invention

The present invention is generally related to the field of orthopedics, and, more particularly, to instrumentation for total knee arthroplasty, and methods of performing same.

2. Description of the Related Art

In the field of orthopedics, total or partial knee replacements are very common. Knee replacement surgery typically involves installing a prosthetic device on the femur of the patient and a prosthetic device on the tibia of the patient. Typically, the distal end of the femur is prepared to accommodate a femoral knee component and the proximate end of the tibia is prepared to accommodate a tibial component. These surfaces are typically prepared by making various saw cuts with the aid of various saw guides. It is very important that the desired cuts are made as precisely as possible as mistakes may lead to poorly performing prosthetic devices and/or require additional surgery to correct a variety of defects.

During a partial or total knee replacement, an incision is made to obtain access to the knee joint. Typically, with some prior art total knee arthroplasty techniques, this incision was relatively long (8″-12″ (203-305 mm)) to enable a variety of instruments, such as alignment guides and resection guides, to be positioned adjacent the distal end of the femur. In accordance with known knee arthroplasty techniques, the incision in the knee of the patient was made while the leg of the patient was extended (straight) and the patient was lying on his back. After the incision was made, the leg was flexed. This position resulted in the soft tissues of the knee being compressed against the back of the knee joint. Various instruments were then used to prepare the femur and tibia for the prosthetic devices to be attached thereto. After surgery was complete, the surgeon would then manipulate the knee to confirm that all of the components and the associated ligaments were properly positioned and tensioned such that the prosthetic device would serve its intended purpose. Thereafter, the knee incision was sutured together. The recuperation period for the above-described knee arthroplasty procedure could be quite long. The trauma associated with such extensive surgery increased the recovery period for the patient.

Recently, efforts have been made to develop minimally invasive total knee arthroplasty procedures wherein a relatively short incision is made to accomplish a total or partial knee replacement, e.g., an incision on the order of approximately 3-5″ (76-127 mm). During such a procedure, it is still necessary to properly size the desired implant components, e.g., a femoral component to be attached to the femur, and to prepare the end surfaces of both the femur and the tibia for prosthetic devices. However, instrumentation that was previously employed in the more invasive knee arthroplasty procedure described above is not readily adaptable for minimally invasive total knee arthroplasty procedures.

FIG. 1 depicts an illustrative femoral sizing/drill guide typically employed with the more invasive total knee arthroplasty procedures described above. As shown therein, a femoral sizing/drill guide 10 is comprised of a body 12, a movable stylus 14, having a tip 16, a plurality of anchoring nails 18, a plurality of protruding feet 24, and a plurality of femur post hole guides 20 that are part of a structure 22. In the more invasive total knee arthroplasty procedures described above, the femoral sizing/drill guide 10 was positioned on a prepared end of a femur (not shown) such that a bottom surface 15 of the guide 10 was flat on the prepared surface of the femur. The protruding feet 24 were positioned against the posterior surface of the femoral condyles. In using the device depicted in FIG. 1, the stylus 14 was moved in an anterior/posterior direction (front of the knee to back of the knee direction) such that the tip 16 would be positioned on the anterior cortex region of the femur. Given the very large incisions employed in the total arthroplasty procedures described above, the anterior cortex region of the femur could be readily accessed, i.e., in a perpendicular manner. The stylus 14 was adapted to move along the rod 17 in one direction, i.e., the anterior-posterior direction, as a portion of the stylus 14 was confined by the slot 13 formed in the body 12 of the guide 10. Anchoring nails 18 were used to initially secure the guide 10 to the prepared end of the femur. Typically, the anchoring nails 18 extended about 0.24″ (6.1 mm) into the femur and within the body 12 which typically had a thickness of approximately 0.5″ (12.7 mm). As indicated in FIG. 1, the two femur post hole guides 20 were part of a single structure 22. Thus, if the surgeon desired to move one of the femur post hole guides, e.g., a little bit lower on the femur in the posterior direction, the other femur post hole guide 20 was moved anteriorly due to the fact that both of the femur post hole guides were part of a single structure 22 and the holes 20 moved as a single unit. The equal and opposite movement of the femur post hole guides mechanism shown in FIG. 1 did not accurately represent true external rotation.

The present invention is directed to various devices and methods for solving, or at least reducing the effects of, some or all of the aforementioned problems.

### SUMMARY OF THE INVENTION

The present invention is directed to instrumentation for total knee arthroplasty, and methods of performing same. In one illustrative embodiment, the device is adapted to be coupled to a prepared end of a femur and comprises a body having a bottom surface and a movable stylus operatively coupled to the body, the stylus having a tip, wherein the stylus is coupled to the body such that the tip may be moved in a direction that is approximately perpendicular to a plane containing the bottom surface.

In another illustrative embodiment, the device comprises a body having a bottom surface, a movable cradle assembly comprising a cross-member and a plurality of shafts slidingly coupling the cross-member to the body, and a stylus operatively coupled to the cross-member.

In yet another illustrative embodiment, the device comprises a body having a bottom surface, a stylus having a tip and means for moving the tip in a direction that is approximately perpendicular to a plane containing the bottom surface of the body. The device further comprises means for moving the tip in a direction that is approximately parallel to the plane containing the bottom surface.

In a further illustrative embodiment, the device comprises a body having a bottom surface, an opening formed in the body, a movable tube positioned in the body and a retractable nail at least partially positioned within the tube, the nail having a shoulder, the nail being adapted to be urged to a position such that an end of the nail extends beyond the bottom surface of the body.

In still a further illustrative embodiment, the device comprises a body having a bottom surface and a plurality of individually positionable drill guides coupled to the body, wherein each of the drill guides may be individually positioned independently of the position of any other drill guide.

In one illustrative embodiment of the present invention, the method comprises making an incision in a patient's knee and attaching a femoral implant sizing guide to a prepared surface of a femur of the patient, the sizing guide having a body having a bottom surface and a movable stylus with a tip. The method further comprises, after the sizing guide is attached to the prepared surface of the femur, moving the tip of the stylus in both a direction that is approximately perpendicular to a plane containing the bottom surface of the sizing device and in a direction that is approximately parallel to the plane containing the bottom surface to position the tip of the stylus at a location proximate an anterior cortex region of the femur and determining a size of a femoral knee prosthesis to be positioned on the femur.

In another illustrative embodiment, the method comprises making an incision in a patient's knee and attaching a femoral implant sizing guide to a prepared surface of a femur of the patient, the sizing guide having a body having a bottom surface, a cradle assembly comprising a cross-member and a plurality of shafts slidingly coupling the cross-member to the body, and a movable stylus operatively coupled to the cross-member, the stylus having a tip. The method further comprises, after the sizing guide is attached to the prepared surface of the femur, moving the cross-member relative to the body to thereby move the tip of the stylus in a direction that is approximately parallel to a plane containing the bottom surface of the sizing guide to position the tip of the stylus at a location proximate an anterior cortex of the femur and determining a size of a femoral knee prosthesis to be positioned on the femur.

In yet another illustrative embodiment, the device comprises making an incision in a patient's knee and attaching a femoral implant drill guide to a prepared surface of a femur of the patient, the drill guide having a plurality of individually positionable femur post hole drill guides. The method further comprises individually positioning at least one of the femur post hole drill guides at a desired location and drilling femur post holes in the femur through the individually positioned post hole guides.

### BRIEF DESCRIPTION OF THE DRAWINGS

The invention may be understood by reference to the following description taken in conjunction with the accompanying drawings, in which like reference numerals identify like elements, and in which:

FIG. 1 depicts an illustrative prior art femoral sizing/drill guide;

FIG. 2 is a perspective view of an illustrative embodiment of a femoral sizing/drill guide in accordance with one illustrative embodiment of the present invention;

FIGS. 3A-3B are enlarged partial views of one embodiment of a telescopic stylus that may be employed with the present invention;

FIGS. 4A-4B are cross-sectional views depicting the retractable nail features of the present invention;

FIG. 5 is an enlarged view of a movable slider incorporating a femoral hole drill guide in accordance with one aspect of the present invention;

FIGS. 6A-6B are sectional views depicting one illustrative manner in which the stylus may be pivotally coupled to a portion of a movable cradle assembly;

FIG. 7 depicts one illustrative embodiment of the femoral sizing/drill guide positioned on a prepared surface of a femur;

FIG. 8 depicts an illustrative femur having femur post holes formed therein using the femoral sizing/drill guide described herein; and

FIGS. 9A-9B are front and back views, respectively, of an illustrative drill guide 120 that may be employed in conjunction with the present invention in some applications.

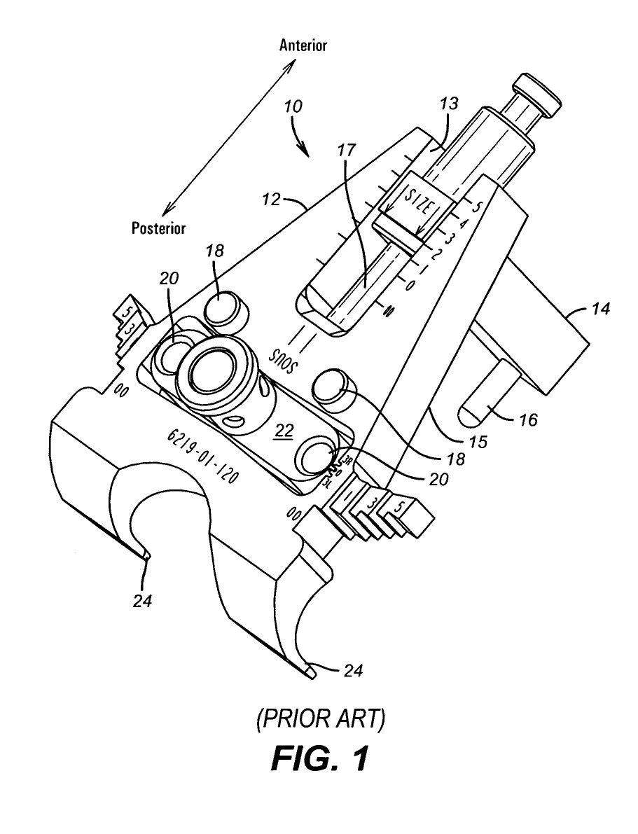

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention will now be described with reference to the attached figures. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase. Various anatomical reference terms used herein are intended to have the standard meaning for such terms as understood in the medical community. For example, the application may include reference to the following terms: anterior (the front, as opposed to the posterior); posterior (the back or behind, as opposed to the anterior); inferior (below, as opposed to superior); superior (above, as opposed to inferior); lateral (toward the left or right side of the body, as opposed to medial); medial (in the middle or inside, as opposed to lateral); proximal (toward the beginning, as opposed to distal); and distal (further from the beginning, as opposed to proximal).

In general, the present invention is directed to instrumentation for total knee arthroplasty, and various methods of performing same. As will be recognized by those skilled in the art after a complete reading of the present application, the present invention may be employed in traditional knee arthroplasty procedures as well as with newer minimally invasive knee arthroplasty procedures. Moreover, the physical configuration of the femoral sizing/drill guide of the present invention disclosed herein, as well as the location and placement of the various features of the device, are provided by way of example only Thus, the particular configuration and arrangement of the features of the femoral sizing/drill guide of the present invention, as well as the particular surgical procedures in which it may be employed, should not be considered a limitation of the present invention, unless such limitations are expressly set forth in the appended claims.

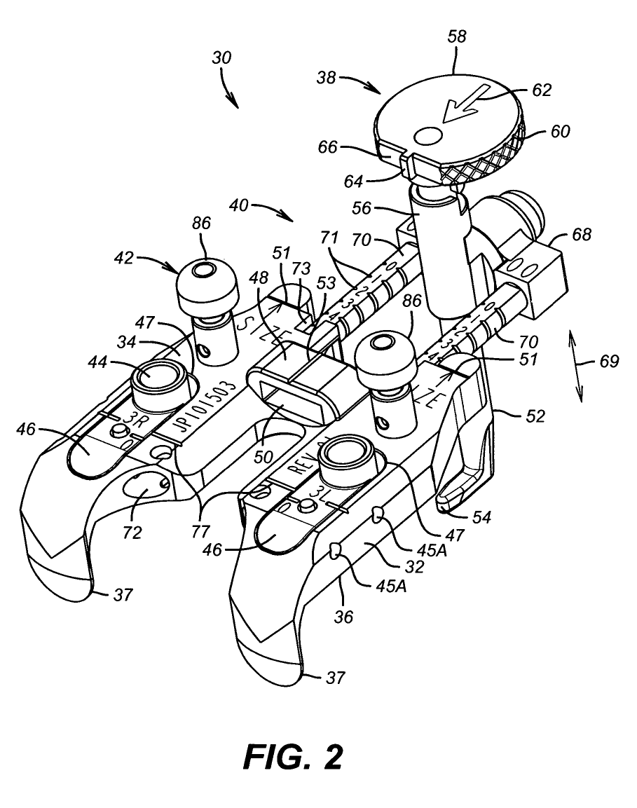

FIG. 2 is a perspective view of an illustrative femoral sizing/drill guide 30 in accordance with one embodiment of the present invention. As shown therein, the device is comprised of a body 32, having a top surface 34, a bottom surface 36, and a plurality of protruding feet 37. The device 30 further comprises a telescopic stylus 38, a telescopic cradle assembly 40, a plurality of retractable nails 42, and a plurality of individually positionable femur post hole drill guides 44 positioned on movable slide bodies 46. The device 30 further comprises an elevated bridge 48 having an opening 50 formed therein. In general, the bottom surface 36 of the device 30 is adapted to be positioned against a prepared surface of a human femur (not shown in FIG. 2) and the protruding feet 37 are adapted to be positioned against the posterior condyles of the femur. The retractable nails 42 will be used to secure the device 30 to the prepared surface of the femur, while the telescopic stylus 38 and telescopic cradle assembly 40 will be used to determine the proper size of a femoral implant that should be installed on the femur of a patient. The drill guide holes 44 will be used to guide the drilling of femur post holes in the femur of the patient. In general, the various components of the device 30 may be made of any of a variety of different biologically compatible metal materials, e.g., stainless steel, cobalt chrome, titanium. The device 30 is intended to be a re-usable device that may be cleaned and sterilized. To that end, the device 30 is provided with a variety of draining and flushing features to insure that the device may be properly cleaned and sterilized.

The telescopic stylus 38 is generally comprised of a shaft 52 having a tip 54. The shaft 52 is positioned within a sleeve 56 and the shaft 52 is adapted to be slidingly and rotatably movable within the sleeve 56. The longitudinal axis of the sleeve 56 is oriented approximately perpendicular to a plane containing the bottom surface 36 of the body 32. The shaft 52 may translate within the sleeve 56 in the direction indicated by the arrows 69. That is, the stylus 38 is operatively coupled to the body 32 such that the shaft 52, and thus the tip 54, may be moved in a direction that is approximately perpendicular to a plane containing the bottom surface 36 of the device 30, e.g., approximately ±5 degrees relative to the plane containing the bottom surface. A handle 58 is operatively coupled to the shaft 52 and the handle 58 may be used to move the shaft 52 laterally within the sleeve 56 and to rotate the shaft 52 and the tip 54. The handle 58 may take any of a variety of shapes. In the depicted embodiment, the handle 58 is a control knob that is eccentrically coupled to one end of the shaft 52. The handle 58 is welded to the shaft 52. The control knob has an exterior knurled surface 60 and a flat surface 66. The control knob further comprises an etched indicating arrow 62 and an indicating projection or bump 64. In general, the indicating arrow 62 and the indicating projection 64 indicate the position of the tip 54. If desired, the stylus 38 may be configured such that it may be stopped at any desired location as the shaft 52 translates the sleeve 56.

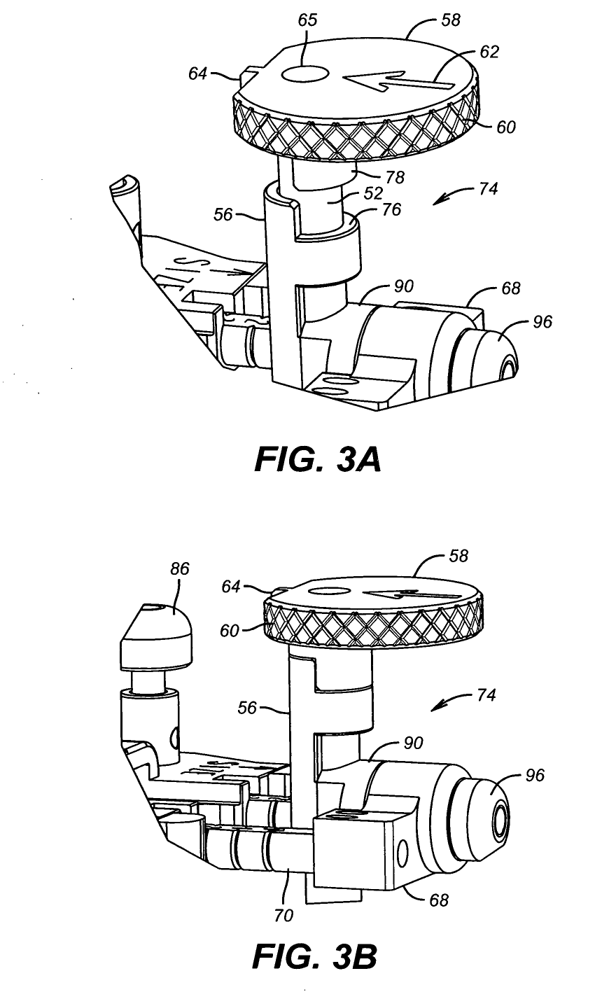

The telescopic stylus 38 further comprises an anti-rotation feature 74 as will be described with reference to FIGS. 3A-3B. In general, the sleeve 56 is provided with a sectional recess 76 and the handle 58 is provided with a sectional projection 78. When the shaft 52 of the telescopic stylus 38 is moved into a position such that the sectional projection 78 on the handle 58 engages the sectional recess 76 in the sleeve 56, the shaft 52 cannot rotate relative to the sleeve 56. FIG. 3A depicts the situation where the shaft 52 is free to rotate within the sleeve 56 whereas FIG. 3B depicts the situation where the anti-rotation feature 74 is engaged, i.e., the sectional projection 78 on the handle 58 engages the sectional recess 76 on the sleeve 56. Of course, those skilled in the art having read the disclosure herein will recognize that there are a variety of different ways to accomplish the objects of the anti-rotation feature 74. Thus, the present invention should not be considered as limited to the precise anti-rotation mechanism depicted in the drawings.

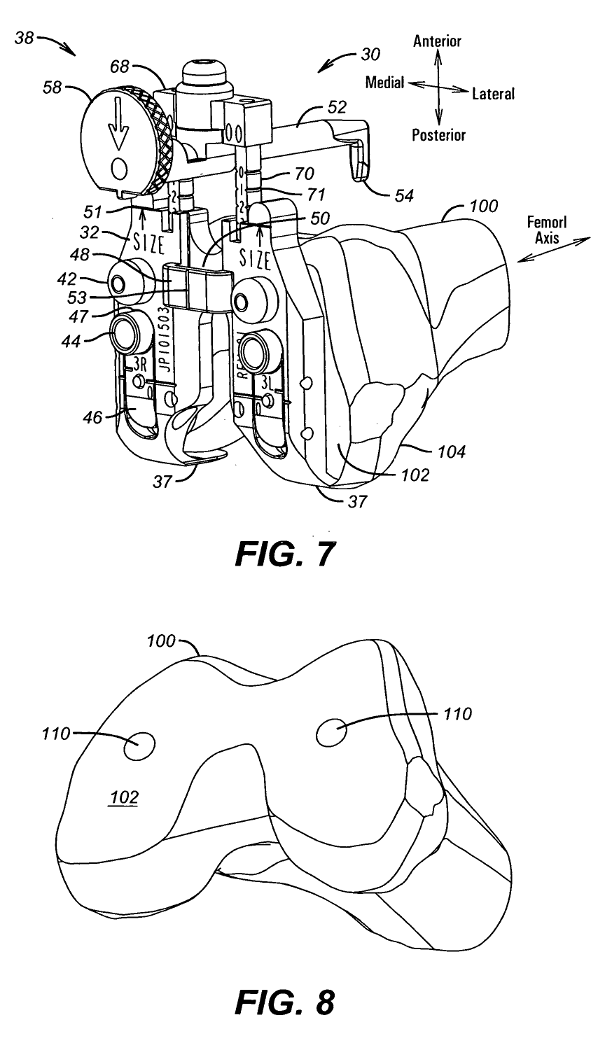

Referring to FIGS. 2 and 7, the telescopic cradle assembly 40 is generally comprised of a cross-member 68 and a plurality of shafts 70. Each of the shafts 70 is adapted to slidingly move within openings 72 formed in the body 32. The shafts 70 are provided with numerical gradations as well as indicating grooves 71 that may be used by the surgeon to determine the proper size of the femoral implant, as will be described later in the application. Lines 51 are formed in the body 32 to assist the surgeon with determining the correct size femoral prosthesis. Windows 73 are also formed in the body 32 such that the surgeon may see how close the next size gradation 71 is to the sizing lines 51.

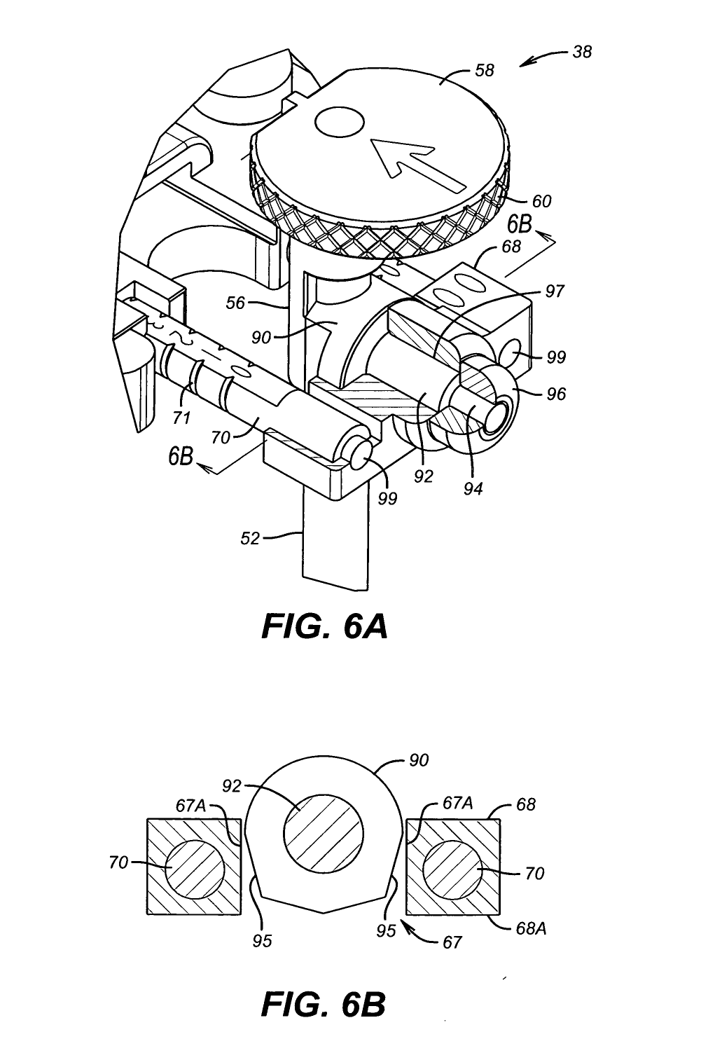

In one illustrative embodiment, the stylus 38 is pivotally coupled to the movable cradle assembly 40. In the depicted example, the stylus 38 is pivotally coupled to the cross-member 68 such that the tip 54 of the stylus 38 may be rotated to a limited degree about the axis of the shaft 52. That is, the stylus is pivotally coupled to the cross-member 68 such that the tip 54 of the stylus 52 may be moved or swept in the medial-lateral direction. FIGS. 6A-6B depict one illustrative technique and configuration for providing such a pivotal connection. As shown therein, the sleeve 56 is generally comprised of a body 90, a sleeve extension 92 and a projection 94. The sleeve extension 92 extends through an opening 97 in the cross-member 68. A lock collar 96 is welded to the projection 94 thereby securing the sleeve 56 within the cross-member 68. The opening 97 is of sufficient size that the sleeve extension 92 may rotate within the opening 97 in the cross-member 68. The shafts 70 are likewise welded to the cross-member 68 at locations 99. As shown in FIG. 6B, the cross-member 68 has a generally rectangular opening 67 formed therein. The opening 67 has a plurality of side surfaces 67A. The body 90 of the sleeve 56 has a plurality of angled surfaces 95 that are disposed at an angle of approximately 8-10 degrees relative to the surfaces 67A of the cross-member 68. The angled surfaces 95 allow the stylus tip 54 of the stylus 38 to be rotated a total range of approximately 16 degrees (±8 degrees) relative to the cross-member 68. Thus, even when the anti-rotation feature 74 is engaged, the tip 54 and the stylus 38 may be rotated approximately 16 degrees (±8 degrees) due to the pivotable connection between the sleeve 56 and the cross-member 68 as described above.

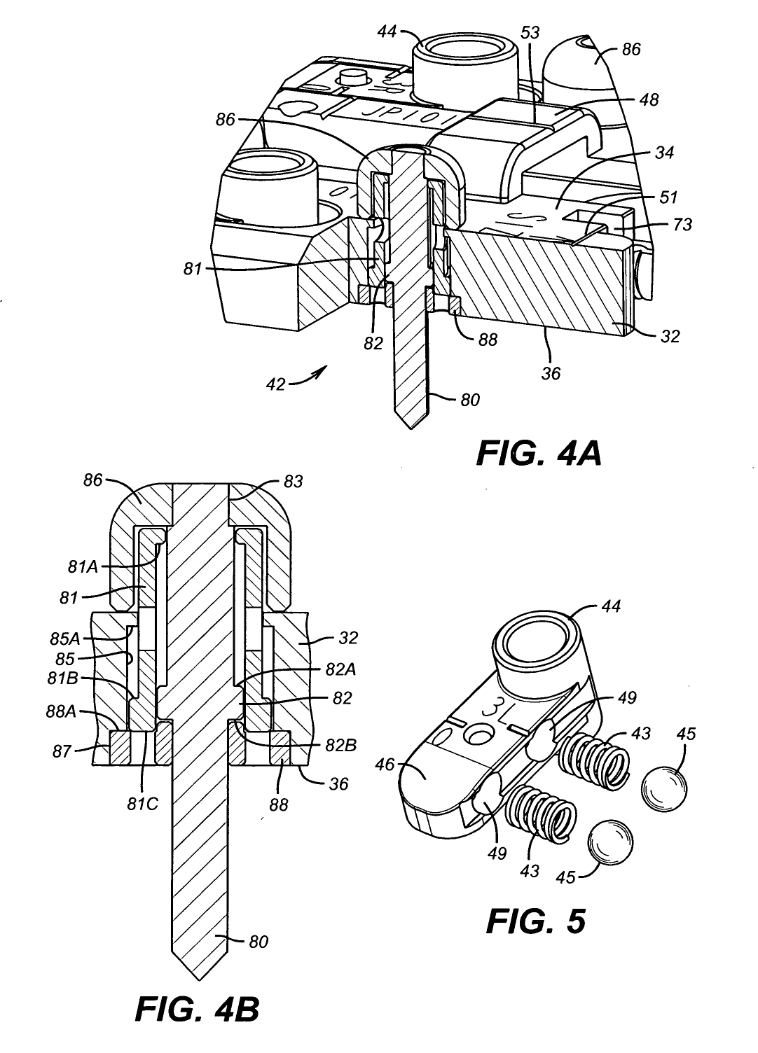

Referring to FIGS. 2 and 4A-4B, the device 30 comprises a plurality of retractable nails 42. In general, the retractable nails 42 will be used to initially secure the device 30 to a prepared surface of the femur. The surgeon may use a variety of methods to drive the retractable nails 42 into the prepared surface of the femur, e.g., a hammer strike or simply push them into the femur. The retractable nails 42 are comprised of a nail 80 having a shoulder 82, a cap 86 and a telescopic or movable tube 81. The cap 86 is welded to the end 83 of the nail 80. The telescopic tube 81 and the nail 80 are retained within an opening 85 formed in the body 32 by a ring 88 that is coupled to the body 32. In one illustrative embodiment, the ring 88 is positioned in a recess 87 formed in the body 32 and welded to the bottom surface 36 of the body 32. The weld is then ground flush such that the bottom surface 36 of the body 32 remains approximately planar. The ring 88 has a top surface 88A. The shoulder 82 on the nail 80 has a top surface 82A and a bottom surface 82B. The telescopic tube 81 has an internal shoulder 81A that is adapted to engage the top surface 82A of the shoulder 82 when the nail 80 is retracted. The telescopic tube 81 further comprises an external shoulder 81B that is adapted to engage an internal shoulder 85A formed in the opening 85. FIGS. 4A-4B depict the nail 80 in the extended position wherein the portion of the nail 80 is extended below the bottom surface 36 of the body 32. This depicts the position of the nail 80 when it is driven into a femur (not shown).

The shoulder 82, the tube 81 and the opening 85 in the body 32 are adapted to interact with one another during the process of extending or retracting the nail 80. Typically, when the surgeon is finished with the device 30, the device may be removed by simply pulling on the device or by using a slide hammer, wherein an end of the slide hammer is positioned in the opening 50 of the elevated bridge 48. Other means are also available for removing the device 30 from the femur after it has served its intended purposes. However, in order to explain the interaction between the nail 80, tube 81 and opening 85, an illustrative retraction sequence will be described. Initially, from the position depicted in FIGS. 4A-4B, the nail 80 is urged upward and moves within the telescopic tube 81 until such time as the top surface 82A of the shoulder 82 engages the internal shoulder 81A of the telescopic tube 81. As the nail 80 continues to move upward, the end 81C of the telescopic tube 81 disengages from the surface 88A of the ring 88. Thereafter, the nail 80 and telescopic tube 81 continue to move upward until the external shoulder 81B on the tube 81 engages the internal shoulder 85A formed in the opening 85, at which time the nail 80 is fully retracted. In one illustrative extension sequence, the nail 80 and the tube 81 are urged downward by pressing or striking the cap 86. The nail 80 and the tube 81 continue to move downward until such time as the external surface 81C on the tube 81 engages the surface 88A of the ring 88. Thereafter, the nail 80 continues to move downward until such time as the bottom surface 82B of the shoulder strikes the surface 88A of the ring 88, at which time the nail 80 is fully extended.

The nails 80 employed in the present invention may be of any desired size. In one illustrative embodiment, the nails 80 have a diameter of approximately 0.125″ (3.2 mm). In general, the retractable nails 42 of the present invention are configured to penetrate into the femur and retain the device 30 in a desired orientation relative to the femur. In one embodiment, the retractable nail feature 42 is configured such that the nail 80, when extended, may extend beyond the bottom surface 36 of the body 32 by a distance of approximately 0.44″ (11.2 mm). However, the nails 80 may be fully retracted, i.e., retracted to the point where the end of the nails 80 do not extend below the bottom surface 36 of the device 30. As will be recognized by those skilled in the art after a complete reading of the present application, the retractable nail features 42 of the device 30 provide a relatively large amount of penetration by the nails 80, e.g., approximately 11 mm, given the reduced overall thickness of the body 32 of the device 30, e.g., approximately 8 mm. The telescopic tube 81 allows for greater stroke, stability and penetration on a much thinner instrument body as compared to prior art devices.

Referring to FIGS. 2 and 5, the device 30 further comprises a plurality of individually positionable femur post hole drill guides 44, each of which are positioned on a movable slide body 46. That is, each of the individually positionable femur post hole drill guides 44 may be positioned at a desired location independent of the positioning of the other hole guides 44. The independent movement of the guide holes 44 offered by the device 30 disclosed herein provides the surgeon with the ability to install a femoral prosthesis with true external rotation. In one illustrative embodiment, the hole guides 44 may have a diameter of approximately ¼″ (6.35 mm). The hole guides 44 are provided with elevated bosses to regulate the drilling depth of a standard collared drill.

FIG. 5 is an enlarged view of an illustrative example of a movable slide body 46 having a guide hole 44 formed therein. In general, the movable slide body 46 is adapted to be movable within a recess 47 formed in the body 32. According to one embodiment, the movable slide body 46 is secured within the recess 47. In the illustrative example depicted in the drawings, this is accomplished using two ball detent mechanisms, each of which is comprised of a spring 43 and a spherical ball 45. The springs 43 and balls 45 are positioned within the openings 49 formed in the movable slide body 46. A portion of the spherical balls 45 engage smaller corresponding receiving holes 45A (see FIG. 2) within the recess 47, thereby slidingly coupling the movable slide body 46 to the body 32. The movable slide body 46 may be moved by simply pushing on the slide body 46 itself. In the depicted embodiment, the hole guides 44 may be positioned at two different locations. As indicated in FIG. 5, the “0” line represents the neutral position of the hole guide 44 whereas the “3R” or “3L” line represents the position of the hole guide 44 with three degrees of external rotation. The body 32 has a plurality of indicator lines 77 formed thereon to aid the surgeon with the proper positioning of the movable slide bodies 46 and thus the hole guides 44. However, as will be recognized by those skilled in the art, the slide body 46 may be slidingly coupled to the device 30 by a variety of techniques. Thus, the illustrative manner depicted in the drawings for slidingly coupling the body 46 to the device 30 should not be considered a limitation of the present invention unless such limitations are expressly recited in the appended claims.

FIG. 7 depicts the device 30 wherein it is attached to a prepared surface 102 of an illustrative femur 100. The protruding feet 37 of the device 30 are engaged with the posterior femoral condyles 104 of the femur 100. As indicated in FIG. 7, the telescopic stylus 38 may be linearly moved in a direction that is approximately perpendicular to a plane containing the bottom surface 36 of the device 30. Stated another way, the device 30 allows the shaft 52 and the tip 54 of the stylus 38 to be moved in a direction that is generally parallel to the femoral axis. The tip 54 of the shaft 52 may be rotated about the axis of the shaft 52 by movement of the control knob 58. The telescopic cradle assembly 40 may be moved in an anterior/posterior direction by virtue of the shafts 70 sliding within the openings 72 formed within the body 32 of the device 30. The movement of the telescopic cradle assembly 40 allows the positioning of the telescopic stylus 38 and its tip 54 in the anterior and posterior direction, i.e., in a direction that is approximately parallel to a plane containing the bottom surface 36 of the device 30. The axial movement of the shaft 52 within the sleeve 56 (in a direction approximately parallel to the femoral axis coupled with the ability to move the shaft 52 (and tip 54) in the anterior-posterior direction by virtue of the telescopic cradle assembly 40 gives the surgeon great flexibility in positioning the tip 54 at the desired location on the anterior cortex region of the femur. Additionally, the pivotal connection between the sleeve 52 and the cross-member 68 enables the surgeon to sweep or rotate the tip 54 in the medial-lateral direction a total range of approximately 16 degrees (±8 degrees from vertical) so as to properly position the tip 54 at the desired location on the anterior cortex region.

As indicated in FIGS. 2 and 7, the device 30 comprises an elevated bridge 48 having an opening 50 formed therein. A groove 53 is provided in the elevated bridge 48 to allow the surgeon to align the groove 53 with various reference marks formed on a prepared end 102 of the femur 100, as described more fully below. In removing the device 30 from the femur, the surgeon may employ a slide hammer device (not shown) wherein an end of a slide rod of the slide hammer is positioned in the opening 50.

As indicated previously, the present femoral sizing/drill guide 30 may be employed with traditional, more invasive knee arthroplasty procedures or with the newer minimally invasive arthroplasty procedures. Thus, the present invention should not be considered as limited to its use with any particular procedure unless such limitations are set forth in the appended claims. The use of the device 30 will now be described with reference to an illustrative surgical process wherein many intermediate steps may be omitted as they are well known to those skilled in the art.

In general, an incision is made to expose a patient's knee joint. An intermedullary rod is inserted into the intermedullary canal of the femur 100. Thereafter, any of a variety of known distal cut blocks (not shown) are positioned on the intermedullary rod. These distal cut blocks are then employed to cut the distal end of the femur to result in the substantially flat prepared surface 102 of the femur 100 depicted in FIG. 7. In preparing the distal end of the femur 100, approximately 10 mm of the femoral condyles are removed. Thereafter, the distal cut block and the intermedullary rod are removed. At this point in the process, the prepared surface 102 of the femur has been formed and it is necessary to determine the appropriate size femoral implant to be attached to the femur 100. To that end, the femoral sizing/drill guide 30 of the present invention may be attached to the prepared surface 102 of the femur by the following process. Initially, the two protruding feet 37 of the device 30 are positioned adjacent and pulled up against the posterior (rear) surface of the femoral condyles 104, as indicated in FIG. 7. In some cases, the patient may not have sufficient cartilage present adjacent the posterior region of the femoral condyles. In such situations, one or more protruding feet 37 may not actually touch the femoral condyle.

The surgeon then positions the device 30 in the correct lateral (in the medial-lateral direction) position on the femur by reference to various reference marks such as a Whitesides line previously marked on the prepared surface 102 of the femur 100. To that end, the device 30 is provided with the groove 53 in the elevated bridge 48 that allows the surgeon to align the underlying reference marks with the groove 53. During the process of laterally positioning the device 30 at a desired location, the nails 80 are in a fully retracted position. Thus, the bottom surface 36 may be moved across the prepared surface 102 of the femur 100 without dragging or catching the device 30. Once the sizing/drill guide 30 is properly positioned, both in a vertical direction and a lateral direction, the retractable nails 42 are driven into the prepared surface 102 of the femur 100 to thereby secure the device 30 on the prepared surface 102 of the femur 100. Again, the present invention allows the nails 80 (see FIGS. 4A-4B) to penetrate approximately 0.44″ (11.2 mm) into the femur. After the device 30 is secured to the prepared end 102 of the femur 100, the surgeon may then position the patient's leg in a near fully extended position, e.g., within approximately 20 degrees of full extension. This is possible due to the very small overall thickness, e.g., approximately 8 mm, of the device 30 disclosed herein.

In the case of a traditional (more invasive) knee arthroplasty procedure, the movable stylus 38 and the telescopic cradle assembly 40 may be manipulated so as to position the tip 54 of the movable stylus 38 adjacent the anterior cortex region of the femur 100. Typically, in the more invasive traditional knee arthroplasty surgery, the incision is of such great length that the anterior cortex region of the femur 100 may be readily accessed from above, i.e., in a perpendicular direction. In a minimally invasive knee arthroplasty procedure, the length of the incision is so small, e.g., 3-5″ (76-127 mm), that the skin and soft tissue of the patient is not removed from above the anterior cortex region of the femur 100. Thus, the tip 54 of the telescopic stylus 38 must be worked underneath the skin and soft tissue of the patient until it reaches the anterior cortex region of the femur 100. According to the present invention, this is accomplished by longitudinally and rotatably manipulating the stylus shaft 52 within the sleeve 56 and by moving the telescopic cradle assembly 40. That is, in a minimally invasive knee arthroplasty procedure, the telescopic stylus 38 and the telescopic cradle assembly 40 are manipulated until such time as the tip 54 is positioned adjacent the anterior cortex region of the femur. Positioning the tip 54 at the desired location on the anterior cortex region typically involves longitudinally extending the shaft 52 within the sleeve 56 until such time as the anti-rotation feature 74 (see FIGS. 3A-3B) between the shaft 52 and the sleeve 56 is engaged. Thereafter, by virtue of the pivotal connection between the sleeve 56 and the cross-member 68, the control knob 58 may be used to rotate or sweep the tip 54 of the stylus 38 within a range of approximately 16 degrees, looking for the lowest point on the anterior cortex of the femur 100. When this lowest point is reached, the gradation lines 71 on the shafts 70 indicate the desired size of the femoral prosthetic implant to be attached to the femur 100. In most cases, a drill may then be inserted through the guide holes 44 to form the femur post holes 110 (see FIG. 8) in the femur 100. Typically, the femur post holes 110 are formed with the guide holes 44 in the neutral position. If a surgeon determines that external rotation of the femoral implant is desired, the position of one or more of the guide holes 44 may be moved prior to forming the femur post holes 110. FIG. 8 is a depiction of the femur 100 after the femur post holes 110 have been formed therein using the device 30 described and depicted herein.

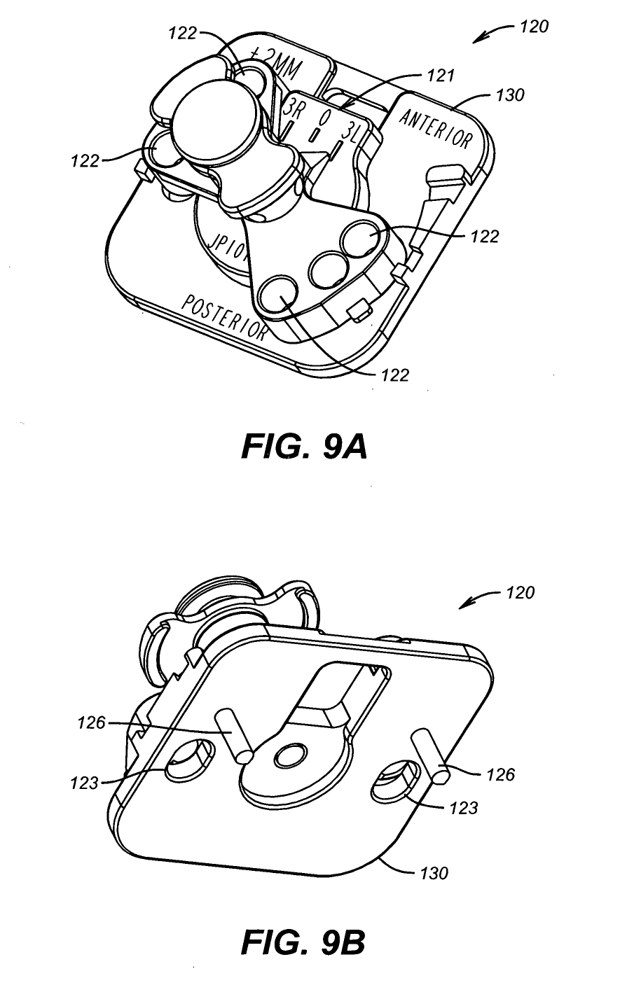

In some cases, the patient's femur 100 may be of such a size that standard size femoral prosthetic devices available on the market do not fit as well as the surgeon would like. That is, while the femoral implant fits properly in a medial-lateral direction, it does not fit as well as the surgeon would like in an anterior to posterior direction. This is a so-called “half-size” situation. In such a case, the surgeon may employ a separate adjustable drill guide 120 depicted in FIGS. 9A-9B to properly locate the femoral holes 110.

In general, in such patients, the surgeon tries to locate the femoral post holes 110 such that the femoral hole pattern is moved anteriorly about 2 mm. The drill guide 120 depicted in FIGS. 9A-9B has six illustrative drill guide holes 122 that may be used by the surgeon in locating the femur post holes in the femur 100. The base 130 of the drill guide 120 only has two holes 123 (see FIG. 9B). However, the holes 123 are of sufficient size and shape to accommodate all of the possible hole pattern configurations that may be drilled using the guide 120. The adjustable drill guide 120 allows the surgeon to select either a neutral position or a 3 degree external rotation for the femur post holes for either the right or left femur. The drill guide 120 has a pointer/scale 121 to indicate the working or desired position of the drill guide holes 122. Only two of the six drill guide holes 122 will line up with the holes 123 in the base 130 of the drill guide 120 at any given position. As shown in FIG. 9B, the drill guide 120 has a plurality of posts 126 that are designed to be positioned in holes in the prepared surface 102 of the femur 100 formed by the nails 80 of the retractable nail feature 42 of the present invention. Once the drill guide 120 is properly located, the surgeon may then drill the desired femur post holes 110 at any desired location and/or external rotation.

After the femur holes 110 are formed in the femur 100, any of a variety of different saw guides are used to form the desired cuts on the end of the femur 100 that correspond to various internal surfaces on a femoral implant. For example, such guides may include a distal surface saw guide, a posterior surface saw guide, an anterior saw guide, a chamfer saw guide and a notching saw guide. Such saw guides are well known in the art and will be described herein in any further detail. Typically, these guides have posts that are adapted to be positioned in the femur post holes 110 previously formed in the prepared surface 107 of the femur 100 using the illustrative device 30 described herein.

The present invention is directed to instrumentation for total knee arthroplasty, and various methods of performing same. In one illustrative embodiment, the device is adapted to be coupled to a prepared end of a femur and comprises a body having a bottom surface and a movable stylus operatively coupled to the body, the stylus having a tip, wherein the stylus is coupled to the body such that the tip may be moved in a direction that is approximately perpendicular to a plane containing the bottom surface.

In another illustrative embodiment, the device comprises a body having a bottom surface, a movable cradle assembly comprising a cross-member and a plurality of shafts slidingly coupling the cross-member to the body, and a stylus operatively coupled to the cross-member.

In yet another illustrative embodiment, the device comprises a body having a bottom surface, a stylus having a tip and means for moving the tip in a direction that is approximately perpendicular to a plane containing the bottom surface of the body, wherein the means for moving the tip in a direction that is approximately perpendicular to the plane containing the bottom surface of the body comprises a sleeve that is adapted to have the stylus positioned therein, the sleeve having an axis that is positioned approximately perpendicular to the plane containing the bottom surface of the body and a handle coupled to the stylus. The device further comprises means for moving the tip in a direction that is approximately parallel to the plane containing the bottom surface, wherein the means for moving the tip in a direction that is approximately parallel to the plane containing the bottom surface of the body comprises a cradle assembly comprising a cross-member, the stylus being operatively coupled to the cross-member, and a plurality of shafts coupled to the cross-member, each of the shafts coupled to the cross-member being slidably positioned within an opening formed in the body.

In still another illustrative embodiment, the device comprises a body having a bottom surface, an opening formed in the body, a movable tube positioned in the body and a retractable nail positioned within the tube, the nail having a shoulder, the nail being adapted to be urged to a position such that an end of the nail extends beyond the bottom surface of the body.

In a further illustrative embodiment, the device comprises a body having a bottom surface, an opening formed in the body, the opening having an internal shoulder, a movable tube positioned in the opening in the body, the tube having an internal shoulder and an external shoulder, and a retractable nail positioned within the tube, the nail having a shoulder with a top surface and a bottom surface, the nail being adapted to be urged to a position such that an end of the nail extends beyond the bottom surface of the body, wherein the top surface of the shoulder on the nail is adapted to engage the internal shoulder on the tube, and the external shoulder on the tube is adapted to engage the internal shoulder of the opening.

In still a further illustrative embodiment, the device comprises a body having a bottom surface and a plurality of individually positionable drill guides coupled to the body, wherein each of the drill guides may be individually positioned independently of the position of any other drill guide.

In one illustrative embodiment of the present invention, the method comprises making an incision in a patient's knee and attaching a femoral implant sizing guide to a prepared surface of a femur of the patient, the sizing guide having a body having a bottom surface and a movable stylus with a tip. The method further comprises, after the sizing guide is attached to the prepared surface of the femur, moving the tip of the stylus in both a direction that is approximately perpendicular to a plane containing the bottom surface of the sizing device and in a direction that is approximately parallel to the plane containing the bottom surface to position the tip of the stylus at a location proximate an anterior cortex region of the femur and determining a size of a femoral knee prosthesis to be positioned on the femur.

In another illustrative embodiment, the method comprises making an incision in a patient's knee and attaching a femoral implant sizing guide to a prepared surface of a femur of the patient, the sizing guide having a body having a bottom surface, a cradle assembly comprising a cross-member and a plurality of shafts slidingly coupling the cross-member to the body, and a movable stylus operatively coupled to the cross-member, the stylus having a tip. The method further comprises, after the sizing guide is attached to the prepared surface of the femur, moving the cross-member relative to the body to thereby move the tip of the stylus in a direction that is approximately parallel to a plane containing the bottom surface of the sizing guide to position the tip of the stylus at a location proximate an anterior cortex of the femur and determining a size of a femoral knee prosthesis to be positioned on the femur.

In yet another illustrative embodiment, the device comprises making an incision in a patient's knee and attaching a femoral implant drill guide to a prepared surface of a femur of the patient, the drill guide having a plurality of individually positionable femur post hole drill guides. The method further comprises individually positioning at least one of the femur post hole drill guides at a desired location and drilling femur post holes in the femur through the individually positioned post hole guides.

The particular embodiments disclosed above are illustrative only, as the invention may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A sizing device that is adapted to be coupled to a prepared end of a femur, comprising:
a body having a bottom surface; and
a movable stylus operatively coupled to said body, said stylus having a tip, wherein said stylus is coupled to said body such that said tip may be moved in a direction that is approximately perpendicular to a plane containing said bottom surface.

2. The device of claim 1, wherein said body further comprises a plurality of protruding feet, each of which is adapted to engage a posterior portion of a femoral condyle.

3. The device of claim 1, wherein said stylus further comprises a shaft that is coupled to said tip, said device further comprising a handle that is operatively coupled to said shaft of said stylus such that said tip of said stylus may be rotated by rotating said handle.

4. The device of claim 1, wherein said tip of said stylus is also movable in a direction that is substantially parallel to said plane containing said bottom surface.

5. The device of claim 1, wherein said stylus further comprises a shaft that is coupled to said tip, wherein said shaft of said stylus is positioned in a sleeve and said sleeve is coupled to a movable cradle assembly, wherein said tip of said stylus is also movable in a direction that is substantially parallel to said plane containing said bottom surface of said body by moving said cradle assembly.

6. The device of claim 1, wherein said stylus further comprises a shaft that is coupled to said tip, wherein said shaft of said stylus is positioned in a sleeve and said sleeve is coupled to a movable cradle assembly that is coupled to said body by a plurality of shafts, each of said shafts coupled to said cradle assembly being adapted to slide within an opening formed in said body, wherein said tip of said stylus is also movable in a direction that is substantially parallel to said plane containing said bottom surface of said body by moving said cradle assembly.

7. The device of claim 1, wherein said stylus has a shaft that is coupled to said tip, said device further comprising:
a cradle assembly, comprising:
a cross-member;
a plurality of shafts coupled to said cross-member, each of said shafts coupled to said cross-member being slidably positioned within an opening formed in said body; and
a sleeve that is pivotally coupled to said cross-member, said shaft of said stylus being positioned within said sleeve.

8. The device of claim 7, wherein said shafts coupled to said cross-member have a plurality of size gradation marks positioned thereon.

9. The device of claim 1, wherein said stylus has a shaft that is coupled to said tip, said device further comprising:
a cradle assembly, comprising:
a cross-member;
a plurality of shafts coupled to said cross-member, each of said shafts coupled to said cross-member being slidably positioned within an opening formed in said body; and
a sleeve that is coupled to said cross-member, said shaft of said stylus being positioned within said sleeve.

10. The device of claim 1, further comprising a plurality of retractable nails positioned in said body wherein, when said nails are in the retracted position, the nails do not extend beyond said bottom surface of said body.

11. The device of claim 1, further comprising a plurality of individually positionable drill guides coupled to said body.

12. The device of claim 11, wherein each of said drill guides is adapted to provide a guide for a femur post hole to be formed in a femur.

13. The device of claim 11, wherein each of said individually positionable drill guides is positioned on a movable body that is positioned in a recess formed in a top surface of said body.

14. A sizing device that is adapted to be coupled to a prepared end of a femur, comprising:
a body having a bottom surface;
a movable cradle assembly, comprising:
a cross-member; and
a plurality of shafts slidingly coupling said cross-member to said body; and
a stylus operatively coupled to said cross-member.

15. The device of claim 14, wherein each of said shafts is adapted to slide within an opening formed in said body.

16. The device of claim 14, wherein said stylus has a tip, and wherein said tip is adapted to be moved in a direction that is approximately parallel to a plane containing said bottom surface of said cradle assembly relative to said body.

17. The device of claim 15, wherein said stylus comprises a tip, and wherein said tip is adapted to be moved in a direction that is approximately parallel to a plane containing said bottom surface by movement of said shafts coupled to said cross-member within said openings in said body.

18. The device of claim 14, wherein said tip may be moved in a direction that is approximately perpendicular to a plane containing said bottom surface.

19. The device of claim 14, wherein said body further comprises a plurality of protruding feet, each of which is adapted to engage a posterior portion of a femoral condyle.

20. The device of claim 14, wherein said stylus further comprises:
a tip;
a shaft that is coupled to said tip; and
a handle that is operatively coupled to said shaft of said stylus such that said tip of said stylus may be rotated by rotating said handle.

21. The device of claim 14, wherein said stylus further comprises:
a tip;
a shaft that is coupled to said tip; and
a sleeve that is pivotally coupled to said cross-member, wherein said shaft of said stylus is positioned in said sleeve.

22. The device of claim 14, wherein said shafts slidingly coupled to said cross-member have a plurality of size gradation marks positioned thereon.

23. The device of claim 14, further comprising a plurality of retractable nails at least partially received within said body wherein, when said nails are in the retracted position, the nails do not extend beyond said bottom surface of said body.

24. The device of claim 14, further comprising a plurality of individually positionable drill guides coupled to said body.

25. The device of claim 24, wherein each of said drill guides is adapted to provide a guide for a femur post hole to be formed in a femur.

26. The device of claim 24, wherein each of said individually positionable drill guides is positioned on a movable body that is positioned in a recess formed in a top surface of said body.

27. A sizing device that is adapted to be coupled to a prepared end of a femur, comprising:
a body having a bottom surface;
a stylus having a tip; and
means for moving said tip in a direction that is approximately perpendicular to a plane containing said bottom surface of said body.

28. The device of claim 27, further comprising means for moving said tip in a direction that is approximately parallel to said plane containing said bottom surface.

29. The device of claim 27, wherein said body further comprises a plurality of protruding feet, each of which is adapted to engage a posterior portion of a femoral condyle.

30. The device of claim 27, wherein said means for moving said tip in a direction that is approximately perpendicular to said plane containing said bottom surface of said body comprises:
a sleeve that is adapted to have said stylus positioned therein, said sleeve having an axis that is positioned approximately perpendicular to said plane containing said bottom surface of said body; and
a handled coupled to said stylus.

31. The device of claim 28, wherein said means for moving said tip in a direction that is approximately parallel to said plane containing said bottom surface of said body comprises:
a cradle assembly, comprising:
a cross-member, said stylus being operatively coupled to said cross-member; and
a plurality of shafts coupled to said cross-member, each of said shafts coupled to said cross-member being slidably positioned within an opening formed in said body.

32. The device of claim 27, further comprising a plurality of retractable nails at least partially received within said body wherein, when said nails are in the retracted position, the nails do not extend beyond said bottom surface of said body.

33. The device of claim 27, further comprising a plurality of individually positionable drill guides coupled to said body.

34. A device that is adapted to be coupled to a prepared surface of a femur, comprising:
a body having a bottom surface;
an opening formed in said body;
a movable tube positioned in said body; and
a retractable nail at least partially positioned within said tube, said nail having a shoulder, said nail being adapted to be urged to a position such that an end of said nail extends beyond said bottom surface of said body.

35. The device of claim 34, further comprising a retaining ring positioned in a recess formed in said body adjacent said bottom surface of said body, said ring being adapted to engage a bottom surface of said shoulder on said nail.

36. The device of claim 34, wherein said tube has an internal shoulder that is adapted to engage an upper surface of said shoulder on said nail.

37. The device of claim 34, wherein said tube has an external shoulder that is adapted to engage an internal shoulder formed in said opening in said body.

38. The device of claim 34, further comprising a plurality of individually positionable drill guides coupled to said body.

39. The device of claim 38, wherein each of said drill guides is adapted to provide a guide for a femur post hole to be formed in a femur.

40. The device of claim 38, wherein each of said individually positionable drill guides is positioned on a movable body that is positioned in a recess formed in a top surface of said body.

41. A device that is adapted to be coupled to a prepared surface of a femur, comprising:
a body having a bottom surface;
an opening formed in said body, said opening having an internal shoulder;
a movable tube positioned in said opening in said body, said tube having an internal shoulder and an external shoulder; and
a retractable nail at least partially positioned within said tube, said nail having a shoulder with a top surface and a bottom surface, said nail being adapted to be urged to a position such that an end of said nail extends beyond said bottom surface of said body, wherein said top surface of said shoulder on said nail is adapted to engage said internal shoulder on said tube, and said external shoulder on said tube is adapted to engage said internal shoulder of said opening.

42. The device of claim 41, further comprising a retaining ring positioned in a recess formed in said body adjacent said bottom surface of said body, said ring being adapted to engage said bottom surface of said shoulder on said nail.

43. The device of claim 41, further comprising a plurality of individually positionable drill guides coupled to said body.

44. The device of claim 43, wherein each of said drill guides is adapted to provide a guide for a femur post hole to be formed in a femur.

45. The device of claim 43, wherein each of said individually positionable drill guides is positioned on a movable body that is positioned in a recess formed in a top surface of said body.

46. A device that is adapted to be coupled to a prepared surface of a femur, comprising:
a body having a bottom surface; and
a plurality of individually positionable drill guides coupled to said body, wherein each of said drill guides may be individually positioned independently of the position of any other drill guide.

47. The device of claim 46, wherein each of said drill guides is adapted to provide a guide for a femur post hole to be formed in a femur.

48. The device of claim 46, wherein each of said individually positionable drill guides is positioned on a movable body that is positioned in a recess formed in a top surface of said body.

49. A method, comprising:
making an incision in a patient's knee;
attaching a femoral implant sizing guide to a prepared surface of a femur of said patient, said sizing guide having:
a body having a bottom surface; and
a movable stylus with a tip;
after said sizing guide is attached to said prepared surface of said femur, moving said tip of said stylus in both a direction that is approximately perpendicular to a plane containing said bottom surface of said sizing device and in a direction that is approximately parallel to said plane containing said bottom surface to position said tip of said stylus at a location proximate an anterior cortex region of said femur; and
determining a size of a femoral knee prosthesis to be positioned on said femur.

50. The method of claim 49, further comprising attaching said femoral prosthesis of said determined size to said femur.

51. The method of claim 49, further comprising rotating said tip of said stylus.

52. The method of claim 49, wherein said femoral implant sizing guide further comprises a plurality of individually positionable femur post hole guides, and wherein the method further comprises:
positioning said individually positionable femur post hole guides at a desired location; and
drilling femur post holes in said femur through said individually positionable post hole guides.

53. A method, comprising:
making an incision in a patient's knee;
attaching a femoral implant sizing guide to a prepared surface of a femur of said patient, said sizing guide having:
a body having a bottom surface;
a cradle assembly, comprising:
a cross-member; and
a plurality of shafts slidingly coupling said cross-member to said body; and
a movable stylus operatively coupled to said cross-member, said stylus having a tip;
after said sizing guide is attached to said prepared surface of said femur, moving said cross-member relative to said body to thereby move said tip of said stylus in a direction that is approximately parallel to a plane containing said bottom surface of said sizing guide to position said tip of said stylus at a location proximate an anterior cortex of said femur; and
determining a size of a femoral knee prosthesis to be positioned on said femur.

54. The method of claim 53, further comprising attaching said femoral prosthesis of said determined size to said femur.

55. The method of claim 53, further comprising rotating said tip of said stylus.

56. The method of claim 53, wherein said femoral implant sizing guide further comprises a plurality of individually positionable femur post hole guides, and wherein the method further comprises:
positioning said individually positionable femur post hole guides at a desired location; and
drilling femur post holes in said femur through said individually positionable post hole guides.

57. A method, comprising:
making an incision in a patient's knee;
attaching a femoral implant drill guide to a prepared surface of a femur of said patient, said drill guide having a plurality of individually positionable femur post hole drill guides;
individually positioning at least one of said femur post hole drill guides at a desired location; and
drilling femur post holes in said femur through said individually positioned post hole guides.

58. The method of claim 57, wherein each of said individually positionable femur post hole drill guides is positionable for locating said femur post holes at a neutral or a 3 degree external rotation position.

59. The method of claim 57, wherein both of said individually positionable femur post hole guides are positioned at a desired location.
